(19) United States
(12) Patent Application Publication
(10) Pub. No.: US 2011/0190733 A1
(43) Pub. Date: Aug. 4, 2011

(54) Through-The-Peg Jejunal Feeding Tube

(76) Inventors: Adrian D'Lima, Deansboro, NY (US); Daniel Vuich, Bloomington, IN (US)

(21) Appl. No.: 13/015,779

(22) Filed: Jan. 28, 2011

Related U.S. Application Data
(60) Provisional application No. 61/301,450, filed on Feb. 4, 2010.

(51) Int. Cl. A61M 39/10

(52) U.S. Cl. 604/539

(57) ABSTRACT

A device for providing access to a living body, comprises a hub including first and second ports configured to couple to external medical devices, the first port including a first lumen extending therethrough along a first axis between a proximal opening at the first port and a distal opening at a distal end of the hub, the second port including a second lumen extending therethrough along a second axis non-parallel to the first axis and intersecting the first axis at a distal end of the second lumen, the distal end of the second lumen being open to the first lumen and an inner sleeve defining a third lumen therethrough from a proximal opening at a proximal end thereof to a distal opening at a distal end thereof in combination with a first connector securing the proximal end of the inner sleeve to the hub at a desired position in the first lumen proximal of the distal end of the second lumen, the first connector being shaped to force an outer surface of the inner sleeve into frictional engagement with an inner surface of the first lumen.

### PRIORITY CLAIM

This application claims the priority to the U.S. Provisional Application Ser. No. 61/301,450, entitled “Endoscope Anchoring Device” filed Feb. 4, 2010. The specification of the above-identified application is incorporated herewith by reference.

### BACKGROUND

A feeding tube is a medical device used to provide nutrition to patients that cannot swallow food. A variety of feeding tubes are used in medical practice. In particular, a PEG-J tube is a combination of an outer PEG (i.e., percutaneous endoscopic gastrodome) tube and an inner jejunal tube that extends through the stomach into the jejunum. The jejunal tube may be used to administer liquefied foods to the patient while the PEG tube facilitates gastric drainage.

### SUMMARY OF THE INVENTION

The present invention is directed to a device for facilitating fluid communication with an internal body structure comprising a hub including first and second ports configured to couple to external medical devices, the first port including a first lumen extending therethrough along a first axis between a proximal opening at the first port and a distal opening at a distal end of the hub, the second port including a second lumen extending therethrough along a second axis non-parallel to the first axis and intersecting the first axis at a distal end of the second lumen, the distal end of the second lumen being open to the first lumen and an inner sleeve defining a third lumen therethrough from a proximal opening at a proximal end thereof to a distal opening at a distal end thereof in combination with a first connector securing the proximal end of the inner sleeve to the hub at a desired position in the first lumen proximal of the distal end of the second lumen, the first connector being shaped to force an outer surface of the inner sleeve into frictional engagement with an inner surface of the first lumen.

### BRIEF DESCRIPTION OF THE DRAWINGS

FIG. 1 shows an perspective view of a device according to a first exemplary embodiment of the present invention;

FIG. 2 shows an exploded perspective view of the device of FIG. 1;

FIG. 3 shows a perspective view of a device according to a second exemplary embodiment of the present invention;

FIG. 4 shows an exploded perspective view of the device of FIG. 3;

FIG. 5 shows a cross-sectional view of a port of the device of FIG. 3; and

FIG. 6 shows a cross-sectional view of another port of the device of FIG. 3.

### DETAILED DESCRIPTION

The present invention may be further understood with reference to the following description and the appended drawings, wherein like elements are referred to with the same reference numerals. The present invention relates to a hub for the delivery and/or removal of fluids and other substances into and out of a living body. In particular, the exemplary embodiments of the present invention describe an improved hub device providing greater tensile strength and secure attachment to a feeding tube. It will be understood by those of skill in the art that although the exemplary embodiments describe a hub device for a PEG-J feeding tube, the present invention may be used for any medical procedure in which it is necessary to deliver and/or remove a fluid or other substances from the body.

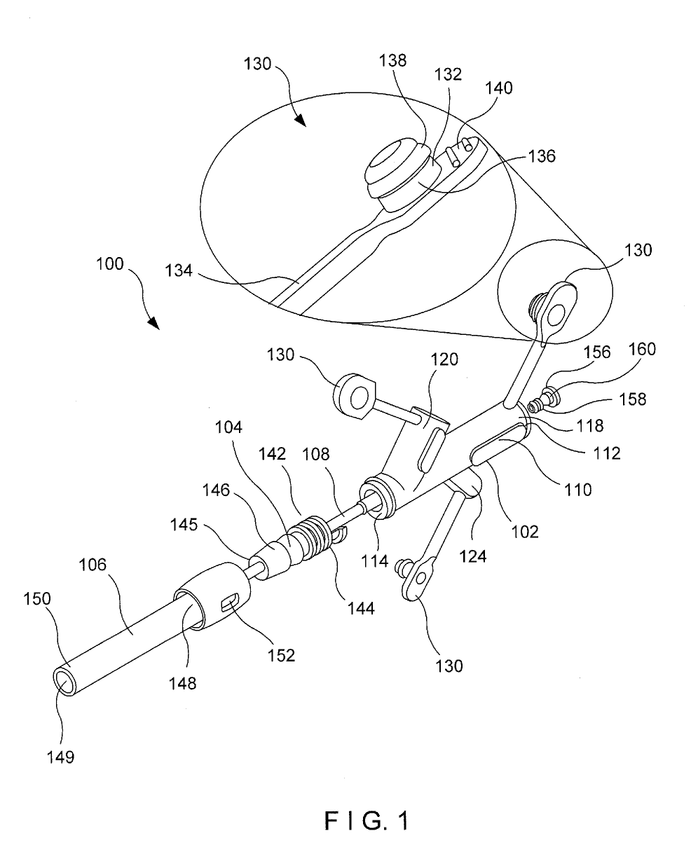

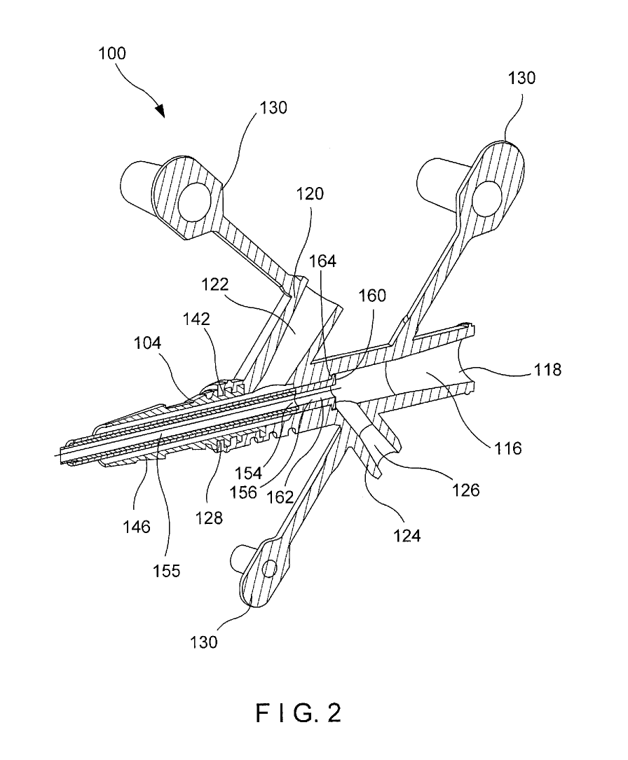

As shown in FIGS. 1-2, a device 100 according to a first exemplary embodiment of the invention comprises a hub 102, a barb connector 104 for connecting to an outer sleeve 106 and an inner sleeve 108 extending through at least a portion of the hub 102 and through the barb connector 104 to extend distally through the outer sleeve 106. The hub 102 includes a body 110 extending from a proximal end 112 to a distal end 114. The body 110 further includes a first lumen 116 extending therethrough, from the proximal end 112 to the distal end 114 such that the a first port 118 at the proximal end 112 is attachable to a pump, syringe or other device for providing fluids, drugs or other substances through the body 110. The hub 102 also includes a second port 120 extending from the body 110 at an angle relative to the body 110, between the proximal and the distal ends 112, 114. The second port 120 defines a second lumen 122 extending therethrough with the second lumen 122 in communication with the first lumen 116. The hub 102 may further include a third port 124 extending from the body 110 at an angle relative to the body 110 and positioned between the first port 118 and the second port 120. The third port 124 defines a third lumen 126 extending therethrough with the third lumen 126 in communication with the first lumen 116. Those skilled in the art will understand that the first lumen 116 extends through the body 110 substantially along a first axis with the second and third lumens 122, 126, extending through the second and third ports 120, 124, respectively, along axes which are non-parallel with respect to the first axis. A distal end of the second lumen 122 intersects with the first lumen 116. Traveling proximally along the second lumen 122 from the distal end, the second lumen 122 diverges from the first lumen 116 so that a proximal opening of the second port 120 is proximal of the distal end thereof and separated from the distal end of the first lumen 116. Similarly, traveling proximally along the third lumen 126 from the distal end thereof, the third lumen 126 diverges from the first lumen 116 so that a proximal opening of the third port 124 is proximal of the distal end thereof and separated from the distal end of the first lumen 116. In this embodiment, the third port 124 extends away from the first axis on a side thereof opposite the second port 120. However, those skilled in the art will understand that the second and third ports 120, 124, respectively, may be arranged in any desired configuration facilitating the connections thereto.

The hub 102 also includes a coupling mechanism 128 at the distal end 114 of the first lumen 116 for coupling with the barb connector 104. The coupling mechanism 128 may include ribs, threadings or any other suitable coupling mechanism as would be understood by those skilled in the art. In a further embodiment, the hub 102 may also include a port cap 130 for any or all of the first, second and third ports 118, 120, 124. Each of the port caps 130 may include, for example, a cap portion 132 and an attaching element 134 coupling each port cap 130 to an outer surface of the corresponding one of the first, second and third ports 118, 120, 124. The cap portion 132 may include a protrusion 136 sized and shaped to be inserted into the first, second and third lumens 116, 122, 126. The protrusion 136 may include an engaging element 138 facilitating engagement of the protrusion 136 with the one of the lumens 116, 122, 126 into which it is to be inserted. The engaging element 138 may be a snap mechanism permitting the protrusion to be snapped within and seal the corresponding lumen 116, 122, 126 to prevent fluids and/or other substances from being passed therethrough. The cap portion 132 may also include a tab 140 located proximately of the protrusion 136 such that the tab 140 may be grasped and pulled by a user to remove the cap portion 132 from the lumens 116, 122, 126. The attaching element 134 permits the port cap 130 to be easily accessed by the user such that the lumens 116, 122, 126 and sealed as desired. Additionally, the attaching element 134 is flexible such that the cap portion 132 may be moved in a variety of different directions to a variety of positions. However, it will also be understood by those of skill in the art that the attaching element 134 is preferably made sufficiently rigid such and biased so that, when the port cap 130 is not in use, the port caps 130 are held in positions that do not restrict access to the ports 118, 120, 124.

The barb connector 104 may include a coupling mechanism 142 at a proximal end 144 thereof for connecting to the hub 102. The coupling mechanism 142 may include, for example, one or more ribs, threading or any other coupling mechanism permitting the barb connector 104 to be locked to the hub 102. In a preferred embodiment, the coupling mechanism 142 includes at least three ribs. It will be understood by those of skill in the art that such a coupling between the barb connector 104 and the hub 102 increases a tensile strength of the device 100 without increasing its overall length and improves resistance to breakage from the bending forces to which it will be subjected. The distal end 144 of the barb connector 104 includes at least a first barb 146 and a second barb 147 for engaging the outer sleeve 106 as will be described in more detail below. In a preferred embodiment, the first barb 146 is at a distal-most end of the barb connector 104. The second barb 147 is proximal of the first barb 146 and includes a length that is approximately 0.25 times the length of the first barb 146 and an outer diameter that is substantially the same as an outer diameter of the first barb 146. This preferred barb connector 104 configuration has been shown to give greater strength in securing the outer sleeve 106.

The outer sleeve 106 may be substantially tubular, extending from a proximal end 148 to a distal end 150 and defining a lumen 149 extending therethrough, such that fluids and/or other substances may pass therethrough. The outer sleeve 106 may be formed of any kind of elastic and compressible polymer tubing such as, for example, silicone. The barb 146 is sized and shaped to be received in the proximal end 148 with a frictional force locking the outer sleeve over the barb 146—i.e., such that the proximal end 148 of the outer sleeve 106 is stretched over the barb 146. A snap ring 152 provides further engagement by, for example, clamping over the proximal end 148 and the barb 146 pinching the proximal end 148 of the outer sleeve about the proximal end of the barb 146 of the barb connector 104. As would be understood by those skilled in the art, the outer sleeve 106 may be placed within the stomach via an endoscopic procedure, such as percutaneous endoscopic gastronomy, in which a user inserts the outer sleeve 106 into the stomach through the abdominal wall. Insertion of the outer sleeve 106 forms an access path via which the inner sleeve 108 may be inserted therethrough, distally past the distal end 150 of the outer sleeve 106 and into the jejunem.

The inner sleeve 108 may also be substantially tubular, extending from a proximal end 154 to a distal end (not shown) with a lumen 155 extending therethrough. In a preferred embodiment, the inner sleeve 108 may be a jejunal feeding tube such that the inner sleeve 108 may be inserted through the outer sleeve 106 and distally therepast through the stomach and into the jejunum. The proximal end 154 may be housed substantially within the hub 102 while a length of the sleeve 108 is selected so that the distal end extends distally through the barb connector 104 and the outer sleeve 106 past a distal end thereof so that, when the outer sleeve 106 is in a desired position opening into the stomach, the inner sleeve 108 may pass out of the outer sleeve 106, through the stomach and into the jejunum. Thus, it will be understood by those of skill in the art that an outer diameter of the inner sleeve 108 is smaller than an inner diameter of the outer sleeve 106 and a length of the inner sleeve 108 is greater than a length of the outer sleeve 106. The outer diameter of the inner sleeve 108 is preferably made small enough that an annular space remains open within the lumen 149 of the outer sleeve 106 around an outer surface of the inner sleeve 108. This annular space may then be used to deliver and/or remove fluids from the stomach even when the inner sleeve 108 extends within the lumen 149. The inner sleeve 108 is preferably formed of a material that permits the inner sleeve 108 to navigate through the stomach and the pylorus to the jejunum. For example, the inner sleeve 108 may be formed from a polypropylene/barium sulfate extrusion such as flexima. It will be understood by those of skill in the art, however, that any kind of elastic and compressible polymer tubing can be used as the sleeve material.

The proximal end 154 of the inner sleeve 108 is adapted and configured to engage a connector 156 connecting the inner sleeve 108 to the hub 102 such that the proximal end 154 remains at a desired position within the hub 102. The connector 156 includes a barbed portion 158 at a distal end thereof for engaging the proximal end 154 of the inner sleeve 108 and forcing this proximal end 154 into a correspondingly shaped recess in the hub 102 to lock the proximal end 154 at a desired position therein. The connector 156 further includes a lumen 162 extending therethrough so that when the barbed portion 158 is received within the proximal end 154 of the inner sleeve 108, fluid may pass therethrough into the inner sleeve 108. The connector 156 also includes a shoulder 160 at a proximal end thereof for engaging the hub 102. The shoulder 160 extends radially outward to engage a shoulder 164 within the lumen 116 preventing the connector 156 from being moved further distally into the lumen 116. It will be understood by those of skill in the art that the connector 156 holds the inner sleeve 108 more securely than a mere adhesive connection between the inner sleeve 108 and the hub 102, which has a higher likelihood of failure. It will also be understood by those of skill in the art that the use of the connector 156 reduces the manufacturing time of the device 100. However, adhesives or other sealing methods may be further utilized to improve the connections of any of the components herein.

The shoulder 164 may be positioned proximally of the point at which the second lumen 122 intersects the lumen 116 and distally of point at which the third lumen 126 intersects the lumen 116 so that the lumen 155 of the inner sleeve 108 communicates only with the first lumen 116 and the third lumen 126. Thus, it will be understood by those of skill in the art that fluids and/or other substances delivered and/or removed via the inner sleeve 108 may only be administered/withdrawn via the first and/or the third ports 118, 124. For example, nutrients to be supplied to the jejunem may be administered via the first port 118 while medication targeted to the jejunem may be periodically administered via the third port 124. The lumen 122 of the second port 120 may then be used for fluids to be withdrawn from or supplied to the stomach.

For example, a drainage pump may be attachable to the second port 120 to drain the stomach via the second lumen 122. Thus, it will be understood by those of skill in the art that each of the first, second and third ports 118, 120, 124 are adapted and configured to be attachable to a variety of medical devices for administering and/or removing fluids or other substances including, for example, syringes, needles, pumps and other drainage and delivery devices. It will also be understood by those of skill in the art that any of the ports 118, 120, 124 may be capped and uncapped as desired to maintain the ports sealed when not in use.

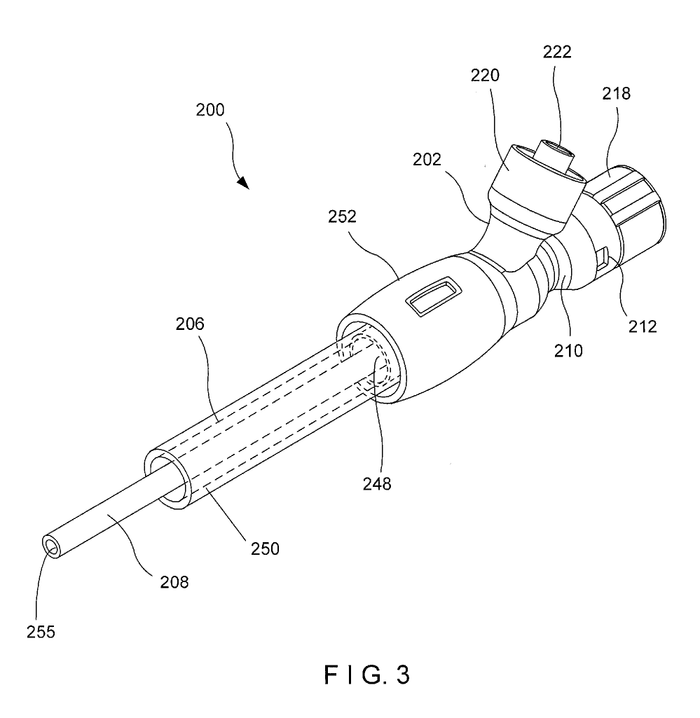

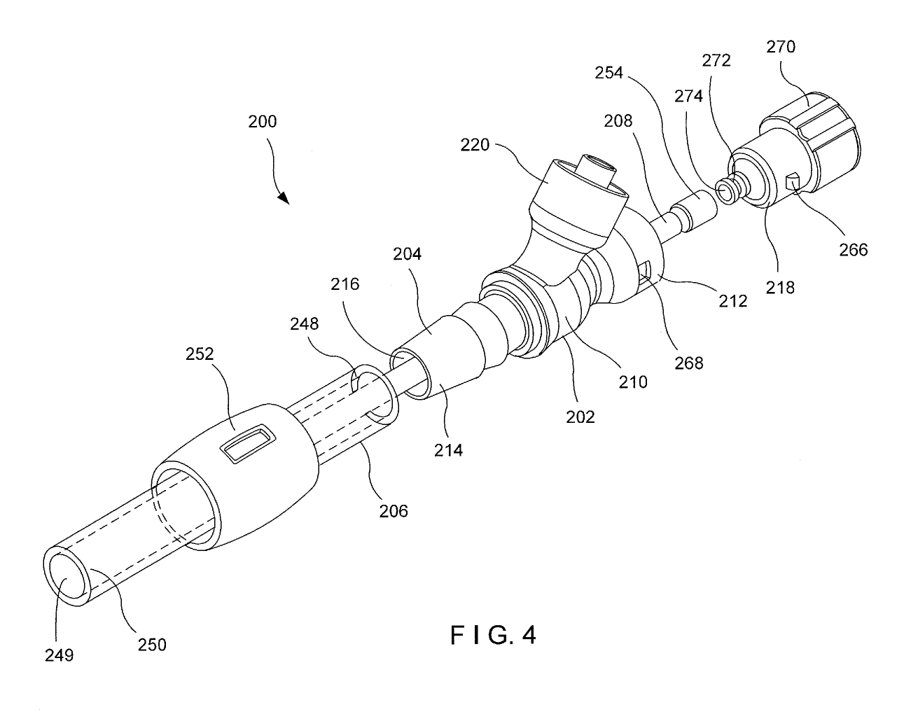

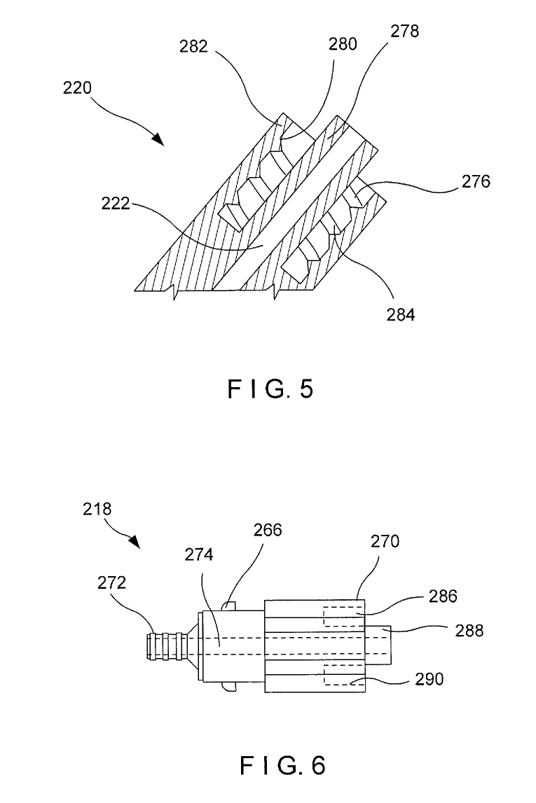

As shown in FIGS. 3-4, a device 200 according to a second exemplary embodiment of the present invention may be substantially similar to the device 100, as described above. The device 200, however, comprises a hub 202 including a barb connector 204 integrally formed at a distal end 214 thereof. Similarly to the device 100, the device 200 further comprises an outer sleeve 206 and an inner sleeve 208. The device 200 further includes a first port 218 couplable with both the hub 202 and the inner sleeve 208. The hub 202 may be substantially similar to the hub 102, including a body 210 extending longitudinally from a proximal end 212 to the distal end 214 with a first lumen 216 extending therethrough. The proximal end 212 is adapted and configured to couple to the first port 218. For example, the proximal end 212 may include a coupling mechanism 268 engagable with a corresponding coupling mechanism 266 of the first port 218. Similarly to the hub 102, the hub 202 also includes a second port 220 extending from the body 210 at an angle relative to the body 210. The second port 220 includes a second lumen 222 extending therethrough such that the second lumen 222 is in communication with the first lumen 216. The second port 220 is adapted and configured to be attached to any of a variety of medical devices to be used therewith (e.g., syringes, needles and pumps) for delivering and/or removing fluids and/or other substances therethrough. For example, as shown in FIG. 5, the second port 220 includes a recess 276 for receiving the medical device, the recess 276 surrounding the second lumen 222. The recess 276 may include a first wall 278 and a second wall 280 that face one another. The first wall 278 may define the second lumen and extend proximally past a proximal end 282 of the second wall 280 such that the first wall 278 may be received within a portion of the medical device. The second wall 280 may, for example, include threading 284 along an inner surface thereof for engaging a correspondingly threaded portion of an medical device to be coupled thereto.

As described above, the first port 218 may be coupled to the hub 202 via the coupling mechanism 266. As shown in FIG. 6, the coupling mechanism 266 may include a protrusion on an outer surface thereof while the coupling mechanism 268 may include a corresponding recess or opening for receiving the protrusion such that the first port 218 engages the hub 202. Alternatively, the coupling mechanisms 266, 268 may be include threading or any other known coupling arrangement to engage one another. The first port 218 and the hub 202 may be coupled such that any fluids and/or substances within the hub 202 will be unable to flow or pass proximally of the proximal end 212 of the hub 202. The first port 218 extends from a proximal end 270 to a distal end 272 and further includes a channel 274 extending therethrough such that when the first port 218 is attached to the proximal end 212 of the body 210, the channel 274 is substantially aligned with and in communication with the first lumen 216. The proximal end 270 is adapted and configured to be coupled to a device for delivering and/or removing substances. For example, similarly to the second port 220, the first port 218 may include a recess 286 with a first wall 288 and a second wall 290, the first wall 288 extending proximally of the second wall 290. The distal end 272 is adapted and configured to engage a proximal end 254 of the inner sleeve 208. The distal end 272 may include a barb or threading thereon (or any other suitable structure) for engaging the proximal end 254 of the inner sleeve 208. Thus, it will be understood by those of skill in the art that fluids administered through the first port 218 will flow directly through a lumen 255 of the inner sleeve 208 and that the inner sleeve 208 can only communicate with the first port 218.

Similarly to the device 100, a proximal end 248 of the outer sleeve 206 is engagable with the barb 204. The barb 204 may be received within the proximal end 248 such that the first and second lumens 216, 222 are in communication with a lumen 249 of the outer sleeve 206. The barb 204 and the proximal end 248 may engage one another via, for example a friction fit. In a further embodiment, a snap ring 252, similar to the snap ring 152, may be clamped over both the proximal end 248 and the barb 204 to further secure the inner sleeve 208 to the barb 204. Since the proximal end 212 of the first lumen 216 is blocked by the first port 218, connected to the inner sleeve 108 and the inner sleeve 208 extends through the lumen 249 of the of the outer sleeve 206, it will be understood by those of skill in the art that a fluid or substance may only be delivered and/or removed through a space within the lumen 249 surrounding the inner sleeve 208 via the second port 220. Thus, a user may administer nutrition and/or medication to the jejunem or withdrawn fluid therefrom through the inner sleeve 208 via the first port 218 and may provide gastric drainage or supply fluids to the stomach through the outer sleeve 206 via the second port 220. It will be understood by those of skill in the art that each of the ports 218, 220 may be adapted and configured to be coupled to any of a variety of devices such as, for example, syringes, needles, pumps, or any other devices capable of delivering or removing fluids and/or other substances.

It will be understood by those of skill in the art that various modifications and variations can be made in the structure and the methodology of the present invention, without departing from the spirit or scope of the invention. Thus, it is intended that the present invention cover the modifications and variations of this invention provided that they come within the scope of the appended claims and their equivalents.

What is claimed is:

1. A device facilitating fluid transfer between an internal structure of a living body and an exterior thereof, comprising:
a hub including first and second ports configured to couple to external medical devices, the first port including a first lumen extending therethrough along a first axis between a proximal opening at the first port and a distal opening at a distal end of the hub, the second port including a second lumen extending therethrough along a second axis non-parallel to the first axis and intersecting the first axis at a distal end of the second lumen, the distal end of the second lumen being open to the first lumen;
an inner sleeve defining a third lumen therethrough from a proximal opening at a proximal end thereof to a distal opening at a distal end thereof; and
a first connector securing the proximal end of the inner sleeve to the hub at a desired position in the first lumen proximal of the distal end of the second lumen, the first connector being shaped to force an outer surface of the inner sleeve into frictional engagement with an inner surface of the first lumen.

2. The device of claim 1, wherein the first connector includes a flared distal end an outer diameter of which is greater than an inner diameter of the third lumen, the flared distal end being inserted into the third lumen and engaging a correspondingly shaped recess in the first lumen to lock the proximal end of the inner sleeve at the desired position in the first lumen.

3. The device of claim 2, wherein the first connector includes an extended diameter proximal end forming a shoulder on an outer surface thereof which, when the first connector is inserted to a desired position in the first lumen engages a shoulder of the first lumen to define a distal-most position of the first connector therein.

4. The device of claim 1, wherein the inner sleeve is formed of a flexible material stretched over the flared distal end of the first connector.

5. The device of claim 1, further comprising:
a second connector a proximal end of which is coupled to a distal end of the hub, the second connector being received around the inner sleeve and including a distal end adapted to couple to a tube through which the inner sleeve extends, the distal end of the second connector including a flared distal end a maximum outer diameter of which is greater than an inner diameter of the tube.

6. The device of claim 1, further comprising:
a third port including a fourth lumen extending therethrough along a third axis non-parallel to the first axis and intersecting the first axis at a distal end of the fourth lumen, the distal end of the fourth lumen being open to the first lumen.

7. The device of claim 6, wherein the third axis intersecting the first axis proximally of the distal end of the second lumen.

8. The device of claim 6, further comprising:
a port cap receivable within at least one of a proximal end of the first port, second port and third port to seal at least one of the first port, second port and third port.

9. The device or claim 5, further comprising:
a snap ring clampable over a portion of the tube that receives the flared distal end of the second connector to secure the tube to the second connector.

10. The device of claim 1, wherein the first connector including a proximal end extending radially outward to contact a correspondingly shaped shoulder within the first lumen, preventing the first connector from moving distally therepast.

11. The device of claim 1, wherein the first connector including a channel extending therethrough substantially along the first axis, the channel being in fluid communication with the first lumen such that an external medical device coupled to the first port is fluidly coupled to the first lumen via the first connector.

12. A method, comprising:
coupling a hub to an inner sleeve via a first connector, the hub including first and second ports, the first port including a first lumen extending therethrough along a first axis between a proximal opening at the first port and a distal opening at a distal end of the hub, the second port including a second lumen extending therethrough along a second axis non-parallel to the first axis and intersecting the first axis at a distal end of the second lumen, the distal end of the second lumen being open to the first lumen, the inner sleeve defining a third lumen such that the first connector locks a proximal end of the inner sleeve at a desired position in the first lumen proximal of the distal end of the second lumen;
inserting a distal end of the inner sleeve into a first target area in a living body; and
coupling an external medical device to one of the first and second ports to deliver a substance to the first target area via the inner sleeve.

13. The method of claim 12, wherein the first connector includes a flared distal end an outer diameter of which is greater than an inner diameter of the third lumen, the flared distal end being inserted into the third lumen and engaging a correspondingly shaped recess in the first lumen to lock the proximal end of the inner sleeve at the desired portion in the first lumen.

14. The method of claim 12, wherein the inner sleeve is formed of a flexible material stretched over the flared distal end of the first connector.

15. The method of claim 12, further comprising:
coupling a tube to the hub via a second connector, the second connector including a proximal end coupling to a distal end of the hub, the second connector being received around the inner sleeve and including a distal end adapted to couple to the tube through which the inner sleeve extends, the distal end of the second connector including flared distal end a maximum diameter of which is greater than an inner diameter of the tube.

16. The method of claim 15, further comprising:
inserting a distal end of the outer sleeve into a second target area of the living body.

17. The method of claim 16, wherein the hub further comprises a third port including a fourth lumen extending therethrough along a third axis non-parallel to the first axis and intersecting the first axis at a distal end of the fourth lumen, the distal end of the fourth lumen opening to the first lumen distal of the proximal end of the inner tube.

18. The method of claim 17, further comprising:
coupling an external medical device to the third port to transfer fluid between the second target area and the medical device.

19. The method of claim 18, further comprising:
inserting a port cap into a proximal end of at least one of the first port, second port and third port to seal at least one of the first port, second port and third port.

20. The method of claim 12, further comprising:
clamping a snap ring over a portion of the tube receiving the flared distal end of the second connector to secure the tube to the second connector.

21. The method of claim 12, further comprising:
coupling an external medical device to the first port via the first connector, the first connector including a channel extending therethrough along the first axis so that the device is in fluid communication with the first lumen.
